(19) United States
(12) Patent Application Publication
(10) Pub. No.: US 2009/0118713 A1
(43) Pub. Date: May 7, 2009

(54) Tissue cleaning apparatus

(75) Inventor: John C. Munson, Easton, PA (US)

Correspondence Address: JONES DAY, 222 EAST 41ST ST, NEW YORK, NY, 10017, US

(73) Assignee: Musculoskeletal Transplant Foundation

(21) Appl. No.: 11/979,532

(22) Filed: Nov. 5, 2007

(51) Int. Cl. A61B 17/00; A61B 19/00

(52) U.S. Cl. 606/1; 128/898

(57) ABSTRACT

A portable tissue cleaning apparatus comprising a housing with a base member and side walls mounted to the base member and a top cover having a motor contained within a housing. A manually operable switch assembly is mounted on the housing and is operable to control operation of the motor. A connector connects the motor to a rotatable basket assembly having a cylindrical container. A perforated basket is mounted in the cylindrical container and a cover mounted on the cylindrical container to provide a sealed fluid containment chamber. The perforated basket is cylindrical and has an impeller mounted on its bottom to direct fluid in a predetermined path when the same is rotated, and a divider comprising a tube with external slots formed therein and fin members mounted in the slots with the fin members extending outward towards an inner wall surface of the cylindrical perforated container to divide the perforated basket into sections. The motor rotatably drives the cylindrical container and perforated basket in sequential clockwise and counter clockwise directions a predetermined number of revolutions to clean tissue held therein.

### RELATED APPLICATIONS

There are no related applications.

### STATEMENT REGARDING FEDERALLY SPONSORED RESEARCH OR DEVELOPMENT

Not applicable.

### REFERENCE TO SEQUENCE LISTING, A TABLE OR A COMPUTER PROGRAM LISTING COMPACT DISC APPENDIX

None.

### FIELD OF THE INVENTION

The present invention relates generally to a tissue cleaning apparatus and more particularly, to a powered rotating perforated basket assembly which is sequentially reversed in clockwise/counterclockwise cycles to clean human tissues.

### BACKGROUND OF THE INVENTION

Several hundred thousand tissue transplants are annually performed in the United States. The single most variable factor with respect to allographic transplantation is the preparation of such bone and tissue segments. Procedure and protocol of the some 400 tissue banks in North America are quite varied and has resulted in various technology with developed processes.

Allografts are vital for bone stock deficiencies that occur during orthopaedic trauma, joint reconstruction, or other reconstructive procedures. The main criteria for an orthopaedic allograft are the retention of strength, the retention of biologic factors, and the reduction of risk of disease transmission. The first two should not be affected by processing, while processing should eliminate the risk for disease transmission.

There is no known industry standard specifying levels of cleanliness for cleaning and preparing bone segments. The problems associated with this lack of standards interpret to poor process control, inadequate removal of tissue from the parent surface and to a large extent lack of sterility during the tissue recovery process.

Human bone obtained from cadaveric donors is typically procured under sterile conditions in an operating suite environment of local hospitals. The bone is stored frozen until it is further processed into small grafts under similar sterile conditions, or under clean-room conditions. Procurement and processing of human tissues is typically performed by groups certified by the American Association of Tissue Banks under standard operating procedures for the processing of each specific bone graft. Large bones such as the femur are thawed and debrided of excess tissue prior to being cut into smaller grafts. Processing of the smaller grafts includes cleaning of bone marrow from the cancellous bone spaces. Cleaning of bone marrow and tissue from small bone grafts has been described in the scientific literature and in brochures and documents made public by groups involved in the procurement and processing of human tissues.

The use of prior art procedures to clean bone tissue involves the use of a pressurized flow of solution as a rapidly moving stream which dislodges bone marrow by impact of the solvent on the bone graft. In U.S. Pat. No. 5,333,626 issued Aug. 2, 1994, a high pressure wash is used to clean bone. The bone is cleaned with a high pressure detergent solution such as TritonX-100 and Tween 80 preferably from 37° C. to 80° C. The bone may be further decontaminated by exposing it to 3% hydrogen peroxide solution from 5 to 120 minutes (preferably 5 to 60 minutes) after which the residual hydrogen peroxide is removed by washing with sterile water. After cleaning, the bone is finally decontaminated by contacting the bone with a global decontaminate for 30 to 60 minutes. Such procedures tend to generate aerosols of tissue and solvent which can be hazardous to processing personnel. The present invention virtually eliminates this hazard.

Ultrasonic cleaners are also used to clean bone tissue. Ultrasonic energy in liquid generated by piezoelectric or other types of transducers creates cavitation, which is the mechanism for ultrasonic cleaning. Cavitation consists of the formation and collapse of countless tiny cavities, or vacuum bubbles, in the liquid. The energy produces alternating high and low pressure waves within the liquid of a tank. The liquid is compressed during the high pressure phase of the wave cycle, then pulled apart during the low pressure phase. As the pressure in the liquid is reduced during the low pressure phase, cavities grow from microscopic nuclei to a maximum critical diameter. During the subsequent high pressure phase they are compressed and implode. The energy is powerful, but safe for parts because it is localized at the microscopic, i.e., cellular, scale. Factors affecting the strength of cavitation are temperature, surface tension, detergents or other agents which reduce surface tension are optimal, viscosity (medium vapor pressure is most conducive to ultrasound activity), and density (where high density creates intense cavitation with greater implosive force).

A number of prior art references have used ultrasonics together with detergents and other solutions to clean bone.

In U.S. Pat. No. 5,556,379 issued Sep. 17, 1996 and U.S. Pat. No. 5,976,104 issued Nov. 2, 1999, the processing of the smaller grafts including cleaning of bone marrow from the cancellous bone spaces using mechanical means, soaking, sonication, and/or lavage with pulsatile water flow under pressure is disclosed. This cleaning may use reduced or elevated temperatures, for example 4° C. to 65° C., and may also include the use of detergents, alcohol, organic solvents or similar solutes or combination of solutes designed to facilitate solubilization of the bone marrow.

In the Simonds reference from the New England Journal of Medicine, page 726, Mar. 12, 1992, entitled TRANSMISSION OF HUMAN IMMUNODEFICIENCY VIRUS TYPE I FROM SERONEGATIVE ORGAN AND TISSUE DONOR, the bone was lyophilized and treated with ethanol. The lyophilized tissue has the soft tissue removed, followed by treatment with two antibiotics, irrigation with sterile water, packaging and refreezing and lyophilization to a residual moisture content of less than 5%. The ethanol treated tissue underwent ultrasonic cleaning in 30% ethanol, removal of marrow by water lavage and brief treatment in 100% ethanol.

U.S. Pat. No. 5,095,925 issued Mar. 17, 1992 is directed toward a bone cleaning device using ultrasonics which removes gross tissue from bone to prepare the same for transplant and use in surgery. The bone is subjected to a positive pressure stream of sterile water, ultrasonically cleaning the same in a detergent followed by rinsing and soaking and reintroduced to the ultrasonic process if necessary within a preferred working temperature range of 27° C. to 33° C.

U.S. Pat. No. 5,509,968 issued Apr. 26, 1996 is directed toward cleaning used orthopaedic implants which are decontaminated and made available for reuse by a three step process for removal of protein tissue, bone tissue and lipids using sonication.

The implant is suspended in an aqueous bath of detergent suitable for emulsifying lipids at elevated temperatures, such as 40° C. to 60° C., and is typically treated for about 1 to 45 minutes by the use of an ultrasonic cleaning system. The solution in the treating container is discarded and the container and implant are washed with clean water. A container is filled with a dilute acid capable of dissolving bone salts (e.g., calcium phosphate minerals that are deposited in the collagen matrix of the bone). The implant is added to the container, and subjected to ultrasonic treatment for approximately the same time. After treatment, the solution containing dissolved bone salts is discarded and the implant and container are again rinsed with clean water. The implant is then subjected to a bath of an aqueous solution sodium hypochlorite of a concentration as sold for general cleaning purposes, (household bleach). This step removes any remaining organic bone tissue as well as protein. An ultrasonic cleaning system is again used for the same time and temperature. When this step is completed, the solution is discarded and both the implant and container rinsed with water.

U.S. Pat. No. 5,797,871 issued Aug. 25, 1998 is also directed toward a bone cleaning process using ultrasonics in which the bone is sonicated in a solution of several detergents within a temperature range of 37° C. to 50° C. to produce bone grafts essentially free from bone marrow and detectable fungal and viral contamination.

A number of prior art patents show the cleaning of bones through agitation and centrifugation.

U.S. Pat. No. 5,513,662 issued May 7, 1996 discloses treating bone at less than ambient pressure and then agitated the same vigorously in an agitator. The U.S. Pat. No. 5,797,871 patent noted above also uses mild and vigorous agitation in connection with its bone cleaning sonication process.

U.S. Pat. No. 5,977,432 issued Nov. 2, 1999 is directed toward a process for removing essentially bone marrow from a cut bone graft. A large substantially intact bone is selected and excess cartilage and associated soft tissues are removed from the surface of the bone. The bone is left whole or may be cut into smaller pieces constituting cut grafts and bone marrow is removed from the cancellous bone spaces of the small cut grafts through the application of centrifugal force. Prior to and/or following the application of centrifugal force, the bone graft may optionally be pretreated with one or more decontaminating agents, and/or solubilizing agents

### SUMMARY OF THE INVENTION

The above and other objects, feature and advantages of the present invention will be apparent in the following detailed description thereof when read in conjunction with the appended drawings wherein the same reference numerals denote the same or similar parts throughout the several views.

The present invention is a portable tissue cleaning apparatus constructed with a housing having a motor contained therein, the housing being provided with a base member, side walls mounted to the base member and a top cover mounted over the housing side walls. A switch assembly is mounted on the top cover for operation of the apparatus. A connector assembly connects the motor to a rotatable perforated basket assembly housing in a fluid container; which is rotated in sequential clockwise and counterclockwise cycles a set number of revolutions to clean bone tissue placed therein. Each sequential cycle is ramped up at the start and ramped down at the end of the cycle. The perforated basket is cylindrical with an impeller mounted on its bottom to direct cleaning fluid in a predetermined path, and the basket is divided into separate sections.

The present invention addresses the needs and deficiencies noted above. It provides an apparatus with a temperature sensor to determine the temperature of the bone cleaning solution.

It is an object of the invention to provide a simple but efficient way of cleaning bones by uniformly cleaning same.

It is another object of the invention that it operates to effectively remove lipids and bone marrow form the cut bone pieces.

It is still another object of the invention that to provide a portable cleaning device which can be easily moved to a desired area.

It is another object of the invention to provide a device that can be simply operated with a minimum of training of personnel.

It is yet another object of the invention that the device can be easily broken apart for sterilization and cleaning.

### BRIEF DESCRIPTION OF THE DRAWINGS

The invention will be better understood and objects other than those set forth above will become apparent when consideration is given to the following detailed description thereof. Such description makes reference to the annexed drawings wherein.

FIG. 1 is a perspective view of the inventive tissue cleaning apparatus;

FIG. 2 is a perspective view of the tissue cleaning apparatus shown in FIG. 1 with the basket assembly and fastening elements for same shown in exploded relationship;

FIG. 3 is an exploded view of the components of the housing of the tissue cleaning apparatus shown in FIG. 1;

FIG. 4 is a perspective view of the basket assembly;

FIG. 5 is a side elevation view of the basket assembly as shown in FIG. 4;

FIG. 6 is an exploded view of the basket assembly shown in FIG. 4;

FIG. 7 is a perspective view of the basket used in the basket assembly of FIG. 4;

FIG. 8 is a side elevation view of the basket shown in FIG. 7;

FIG. 8a is an enlarged side elevation view of the basket mounting taken from Circle A of FIG. 8;

FIG. 9 is an exploded view of the basket shown in FIG. 7;

FIG. 10 is a top plan view of the impeller of the basket assembly shown in FIG. 7;

FIG. 11 is an enlarged cross section taken from line 11′-11′ of FIG. 10;

FIG. 12 is a bottom perspective view of the impeller shown in FIG. 10;

FIG. 13 is a top perspective view of the impeller shown in FIG. 10; and

FIG. 14 is an enlarged view taken from Circle B of FIG. 13.

### DETAILED DESCRIPTION OF THE INVENTION

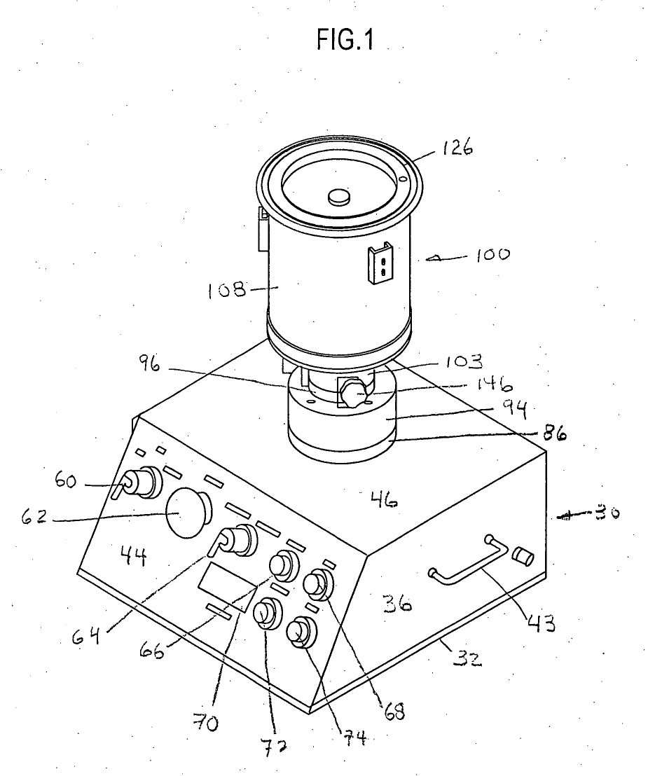

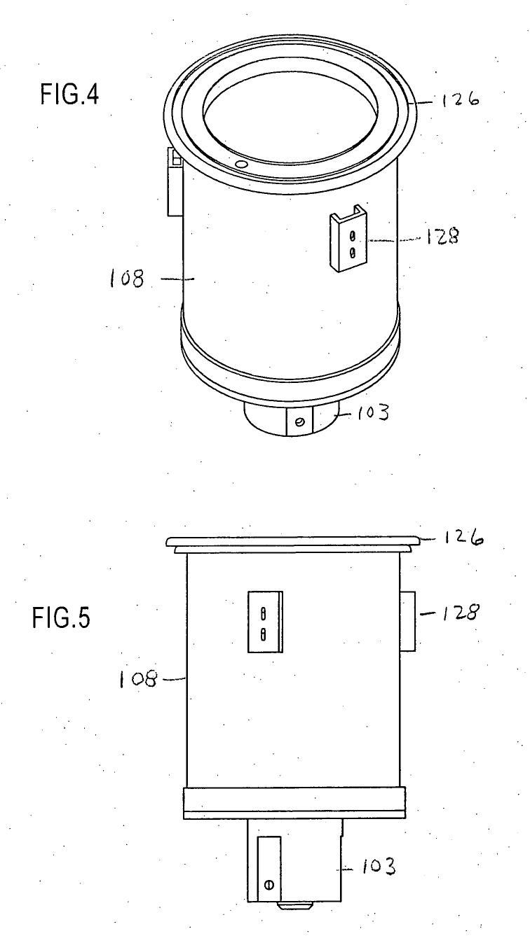

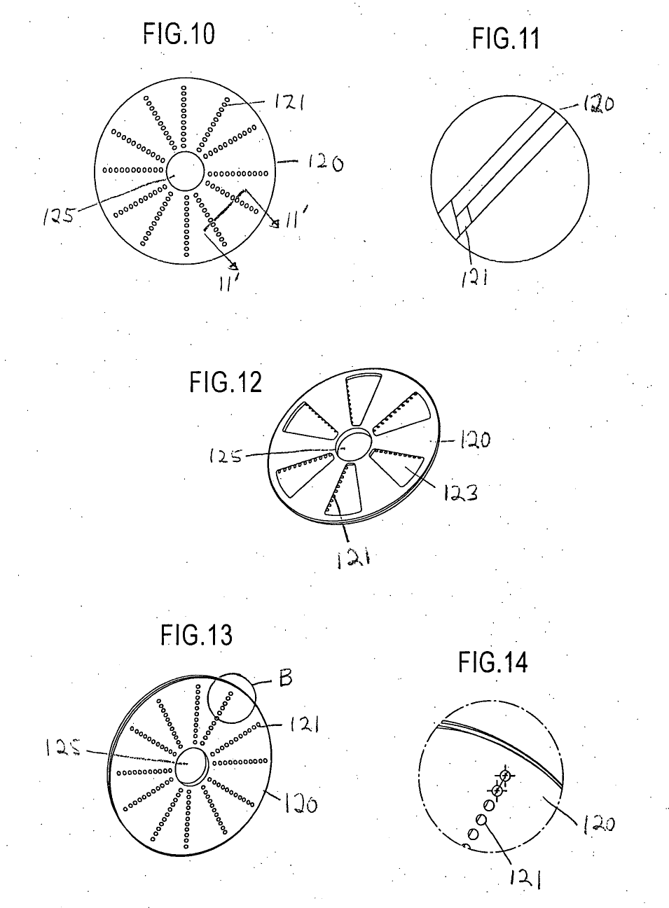

The preferred embodiment and best mode of the invention is shown in FIGS. 1 through 14. While the invention is described in connection with certain preferred embodiments, it is not intended that the present invention be so limited. On the contrary, it is intended to cover all alternatives, modifications, and equivalent arrangements as may be included within the spirit and scope of the invention as defined by the appended claims.

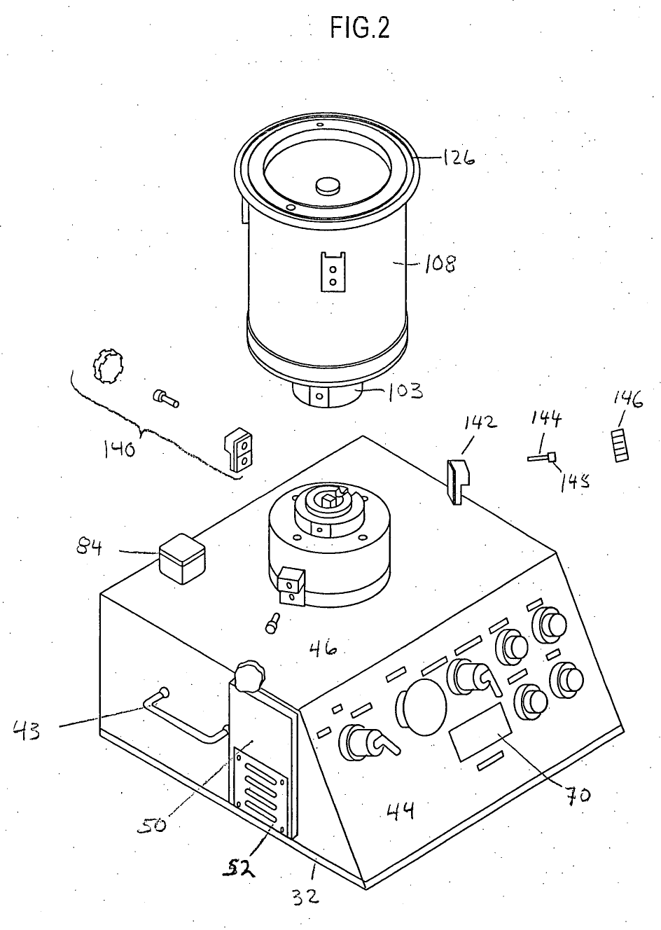

The present invention generally refers to a portable tissue cleaning apparatus 20 constructed with a drive and control housing 30 having a basket assembly 100 mounted thereon. The basket assembly 100 is mounted on the bearing assembly 94 of the servo motor by three mounting bracket assemblies 140 positioned equal distant around the circumference of bearing assembly 94 and basket bearing housing 103 of the basket assembly 100. Each mounting bracket assembly 140 is constructed with a mounting bracket 142, a 5/16×0.875 inch screw 144 and a 5/16 inch rosette thumb screw knob 146 which fits over the head 145 of the screw 144 as is shown in FIG. 2. The bracket assembly 140 is mounted to the basket bearing assembly 103 and the servo motor bearing housing 94.

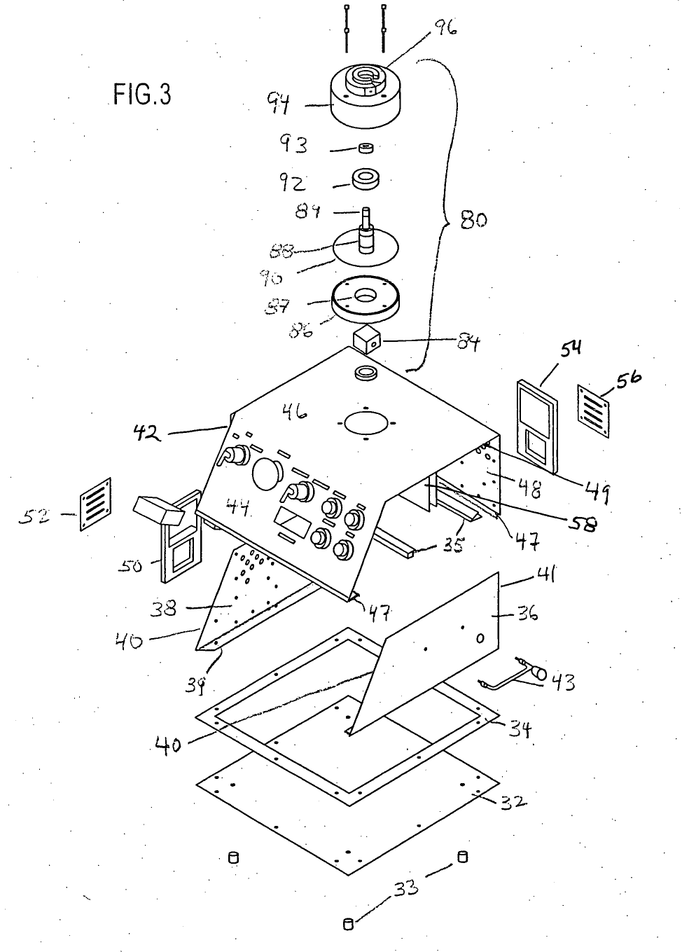

The housing 30 as shown in exploded view in FIG. 3 is formed with a flat rectangular shaped bottom plate 32 having seating bumpers 33 secured thereto, a rectangular gasket 34 which is placed on the top surface of the bottom plate 32 and is secured around the outer periphery of the bottom plate 32 by screws and/or adhesives. Side panels 36 and 38 are formed with a perpendicular lip 39 which is seated on the gasket 34 and held in place by screws. Each of the side panels 36/38 are trapezoidal shaped with angled front edge 40 having a 60° angle from the base and a perpendicular rear edge 41 and have a flat surface. Side panel 38 has a plurality of holes cut therein to allow for ventilation of the interior of the housing. Reinforcing ribs 35 about each of the side panels to provide housing stability. Handles 43 are mounted to the flat surface of each of the side panels 36 and 38 to allow the device to be easily moved from one area to another. Mounted to side panel 38 over the plurality of ventilation holes is a ventilation base enclosure member 50 with a louver 52 for ventilation of the interior of the housing 30. A top cover panel 42 is mounted over the side panels 36 and 38 with the front section 44 of the cover panel 42 having the same angle as the front edge of the side walls 36/38 and a top section 46 being planar and parallel to the bottom plate 32, with the rear section 48 being perpendicular and fitting over the rear edges 40 of the side panels 36/38. Both front section 44 and rear section 48 have an inwardly projecting lip 47 allowing the top cover panel to be mounted on gasket 34 and secured to bottom plate 32. The rear section 48 has a plurality of holes 49 cut in the upper surface of the section to allow ventilation. The bottom plate 32, side panels 36/38 and top cover panel 42 are preferably constructed of stainless steel. Mounted to the rear section 48 of the top cover panel 42 is a ventilation base enclosure member 54 with a louver 56 allowing the interior of the housing to be ventilated. A tube axial fan 58 is mounted to the rear of the rear panel 42 to assist in circulation of the air within the housing.

The front section 44 has a plurality of holes cut therein in a linear alignment to receive a number of controls to operate the cleaning apparatus. As can be seen in FIGS. 1 and 2, a three position select power switch 60 is located on the far left of the front section with an emergency stop button 62 being located next to the switch 60. Switch 60 is a main power switch with an off/on/reset select position. The reset of switch 60 is used to reset servo drive faults. The stop button 62 is used as an emergency stop. Next to the stop button 62 is a two position select switch 64. This switch has a preset 1 and a preset 2 allowing for different servo motor profiles. Currently preset 1 and preset 2 are both set at 1000 rpm but if desired they can be programmed for different speeds. A green start push button 66 is located adjacent the two position select switch 64 and is used to start the apparatus. Next to the green push button 66 in the same linear alignment is a red stop push button 68 which is used to stop the a device. Located beneath the two position select switch 64 is a temperature switch 70 which indicates current temperature. This allows authorized personnel to change temperature at which the machine cycle is disabled and adjacent to the temperature switch is a red fault indicator 72 which is an alarm indicator indicating a fault with the servo drive. Adjacent the red fault indicator 72 is a temperature indicator 74 which indicates that the cleaning solution temperature has reached the preset value. An electronic control card which operates the servo motor in response to the actuation of the various switches and buttons is mounted on the inside of the housing.

A servo motor mount assembly 80 is mounted on the top section 46 and is connected to the basket bearing assembly 103 for driving the basket assembly 100. The servo motor mount assembly 80 is constructed with a 1⅛ inch buna-n u-cup 82, a junction box assembly 84, and a servo mount base 86 defining a central aperture 87. A coupling member 88 is mounted in central aperture 87 of the servo mount base 86 and an o ring 90 is placed around the periphery of the servo mount base 86 to provide a seal with the servo mount bearing housing 94. The coupling has a solid square shaped distal drive end 89. A double sealed ball bearing race 92 is seated over coupling member 88 and is held in place by a second ⅝ inch buna-n u-cup 93. A servo motor mount bearing housing 94 is mounted to the coupling member 88. The servo mount bearing housing 94 is provided with a hub 96 which receives the basket assembly 100. The bearing housing 94 is mounted to the coupling member 88.

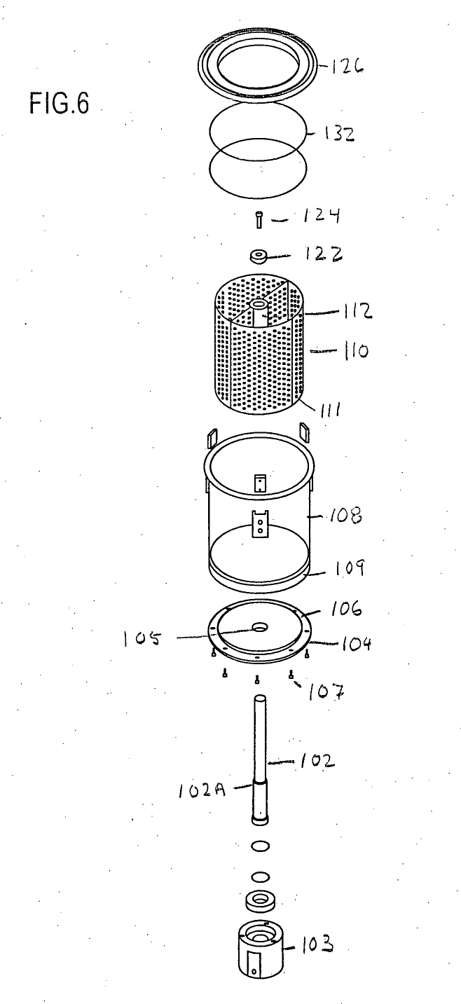

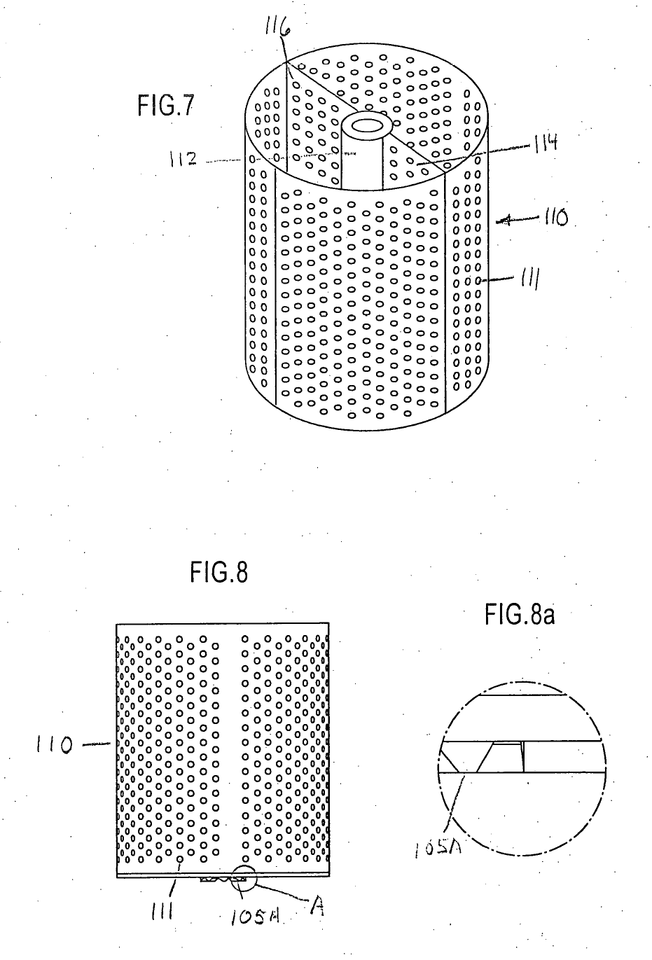

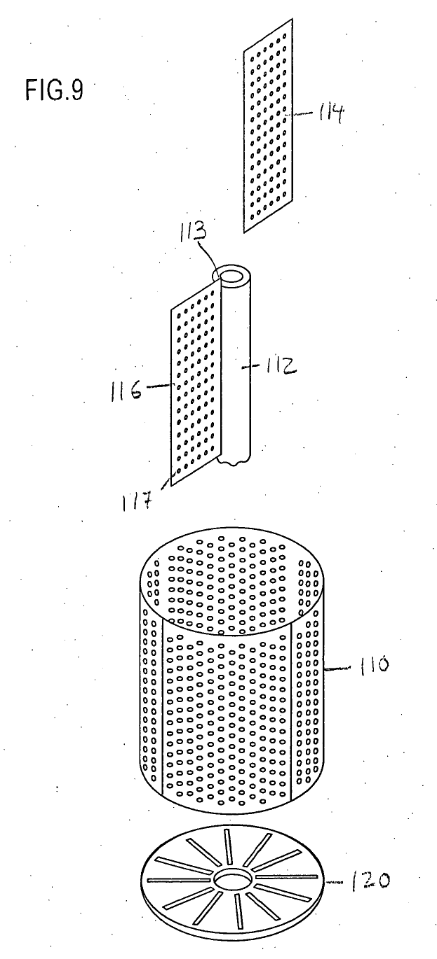

The basket assembly 100 which is shown in exploded view in FIG. 6 is mounted to the hub 96 of the servo mount. A connector shaft 102 is mounted in the basket bearing housing 103 which is mounted by mounting brackets 140 to the servo motor hub 96. The connector shaft 102 extends through an aperture 105 cut in a base plate 104 of the basket assembly. The base plate 104 has a stepped recess 106 cut around its periphery which holds the end 109 of cylindrical container 108 and has a toothed bottom hub 105(a) around aperture 105 as shown in FIGS. 8 and 8a to receive stepped teeth 102a formed on connector shaft 102. The base plate 104 and cylindrical container 108 are secured together by screws 107 which are mounted through equal distant spaced holes in the stepped recess 106 and threaded into threaded holes cut in the end rim 109 of the cylindrical container 108. A perforated basket 110 is mounted in the cylinder container 108 to hold the tissue to be cleaned. The basket 110 has linear rows of holes 111 which are alternatively staggered. The perforated basket 110 which is shown in enlarged view in FIG. 7 and exploded view in FIG. 9 is cylindrical and is divided into two sections by a central positioned tube 112 through which connector shaft 102 is mounted and two opposed separator fin members 114 and 116. The fin members are mounted into slots 113 cut into the tube 112 as is shown in FIG. 9. The separator fin members 114 and 116 extend outward from the tube 112 exterior surface until they engage the inner surface of the cylindrical perforated basket 110. The separator members 114 and 116 are perforated with a plurality of linearly aligned holes 117 having a diameter ranging from 0.18 to 0.20 inches. The base of the perforated basket 110 has an impeller 120 as shown in FIGS. 9 through 14. The impeller 120 is disc shaped and has a plurality of linear radially extending rows of angled through going holes 121 and trapezoidal shaped cutouts 123 on one face. The cutouts have sidewalls which are aligned with two rows of the holes 121 to form 30° wedge segments. The holes have a preferred diameter of about 0.125 inches. The impellor has a central hole 125 which allows connector shaft 102 to be inserted there through. An end cap member 122 fits into the end of tube 112 holding the basket in a fixed axial position on the shaft 102. The end cap member 122 is held in place on shaft 102 by means of a screw 124 which is mounted through the end cap member 122 into the end of shaft 102. A container cover 126 is mounted over the cylindrical container 108 and held in place on the cylindrical container 108 by clamp members 128 which are mounted on clamp blocks 130 secured to the outer surface of the cylindrical container 108. The container cover 126 is stepped and the top of the cylindrical container is sealed by an o-ring 132.

In operation the bone tissue to be cleaned is placed into perforated basket 100 that has fin dividers 114 and 116. As the basket spins, the tissue is forced to spin in the same rate as the basket 100. The basket 100 has an impeller 120 at the bottom which forces cleaning solution up through the basket 100 and promotes fluid circulation over the tissue. Two liters of cleaning solution such as 0.1% Triton X-100 or 0.1% Tween 80 is used per cycle. The basket 100 has a ramped acceleration and deceleration which prevents the tissue from being damaged. The basket spins at a speed of 1000 rpm for 420 revolutions, then stops and spins in the opposite direction for 420 revolutions (equal to 25 seconds CW, 25 seconds CCW). The CW/CCW cycle continues until the operator stops the machine or until the cleaning solution reaches a temperature of 104° F. The inherent temperature rise of the cleaning solution at current setting (1000 rpm, 420 revolutions in the CW and CCW directions) is 0.6° F. per minute. The temperature of the cleaning solution is monitored to ensure that the machine shuts down if 104° F. is reached to prevent temperature damage of the BMP's and growth factors of the tissue. The number of revolutions that the basket 100 turns in each direction is programmable. The direction is reversed to reposition the tissue to ensure even cleaning of all tissue and helps eliminate the possibility that the tissue and cleaning solution reach the same speed. If the tissue and cleaning solution travel at the same speed and in the same direction, there will not be a washing effect as it would be more of a soaking effect. The basket 100 is removable from the cylindrical container and the tissue is removed from the basket for further processing. The basket 100 is autoclaved between each donor tissue usage to ensure sterility and donor separation.

The principles, preferred embodiments and modes of operation of the present invention have been described in the foregoing specification. However, the invention should not be construed as limited to the particular embodiments which have been described above. Instead, the embodiments described here should be regarded as illustrative rather than restrictive. Variations and changes may be made by others without departing from the scope of the present invention as defined by the following claims:

What we claim is:

1. A portable tissue cleaning apparatus comprising:
a housing having a drive means contained within said housing;
a switch assembly mounted on said housing being operable to control operation of said drive means;
a connector means connecting said drive means to a rotatable basket assembly;
said basket assembly comprising a cylindrical container mounted on said connector means, a perforated basket mounted in said cylindrical container, an impeller mounted to said perforated basket and a cover mounted on said cylindrical container to provide a sealed fluid containment chamber;
said drive means rotatably driving said cylindrical container in sequential clockwise and counter clockwise directions a predetermined number of revolutions to clean tissue held therein.

2. The apparatus of claim 1 wherein said housing has a base member, side walls mounted to said base member and a top cover mounted over said housing side walls

3. The apparatus of claim 1 wherein said perforated basket is cylindrical with an impeller mounted on its bottom to direct fluid in a predetermined path, said impeller defining a plurality of linear rows of holes extending radially outwardly.

4. The apparatus of claim 1 wherein said plurality of linear rows comprises 12 rows.

5. The apparatus of claim 3 wherein divider means is mounted in said cylindrical perforated basket to divide the basket into separate sections, said divider means comprising a tube with external slots defined therein and fin members mounted in said slots, said fin members extending outward towards an inner wall surface of said cylindrical perforated basket.

6. The apparatus of claim 5 wherein said fin members have a rectangular configuration and are perforated.

7. The apparatus of claim 1 wherein said impeller is disc shaped and has a plurality of linear rows of radially extending apertures formed therein and a plurality of spaced wedge shaped recesses formed on one side of said disc.

8. The apparatus of claim 1 wherein said manually operated switch assembly comprises a start button, a stop button, a switch for preset servo motor profiles, and an internal solution temperature indicator.

9. The apparatus of claim 8 wherein said preset servo motor profiles are both set at 1000 rpm.

10. The apparatus of claim 7 wherein said temperature indicator disables said apparatus with the temperature reaches 104° F.

11. The apparatus of claim 1 wherein said housing has at least one side wall which defines a plurality of ventilation apertures therein.

12. The apparatus of claim 1 wherein a fan is mounted to said housing.

13. The apparatus of claim 2 wherein said fan is a tube axial fan.

14. The apparatus of claim 1 wherein said housing has handles mounted to said side walls.

15. The apparatus of claim 1 wherein said cylindrical container has a removable base plate secured thereto and said connector means comprises a shaft mounted to a servo bearing housing extending through said base plate.

16. A portable tissue cleaning apparatus comprising:
a housing having a motor contained within said housing; said housing being formed with a base member, side walls mounted to said base member and a top cover mounted over said housing side walls
a manually operable switch assembly mounted on said housing being operable to control operation of the motor;
a connector means connecting a drive mechanism of said motor to a rotatable basket assembly;
said basket assembly comprising a cylindrical container mounted on said connector means, a perforated basket mounted in said cylindrical container, a cover mounted on said cylindrical container to provide a sealed fluid containment chamber, said cylindrical perforated basket including an impeller mounted on its bottom to direct fluid in a predetermined path, and divider means mounted in said perforated basket to divide the basket into sections; said divider means comprising a tube with fin members mounted in said tube, said fin members extending outward towards an inner wall surface of said cylindrical perforated container;
said motor rotatably driving said cylindrical container in sequential clockwise and counter clockwise directions a predetermined number of revolutions to clean tissue held therein.

17. The apparatus of claim 16 wherein said switch assembly impeller has a plurality of linear rows of radially extending apertures formed therein and a plurality of spaced wedge shaped recesses formed on one side.

18. The apparatus of claim 16 wherein said cylindrical container has clamp means secured thereto to hold a cover member in a secured relationship.

19. A portable tissue cleaning apparatus comprising:
a housing having a motor contained within said housing; said housing has a base member, side walls mounted to said base member and a top cover mounted over said housing side walls a switch assembly mounted on said housing being operable to control operation of the motor;
said switch assembly including a start switch, a stop switch and a switch for preset motor profiles;
a connector means connecting said motor to a rotatable basket assembly;
said basket assembly comprising a cylindrical container mounted on said connector means, said cylindrical container having a removable base plate secured thereto, a perforated basket mounted in said cylindrical container and a cover mounted on said cylindrical container to provide a sealed fluid containment chamber; said perforated basket is cylindrical with an impeller mounted on its bottom to direct fluid in a predetermined path, and has divider means mounted in said cylindrical perforated basket to divide the basket into sections; said divider means comprising a tube with external slots formed therein and fin members mounted in said slots, said fin members extending outward towards an inner wall surface of said cylindrical perforated container;
said motor rotatably driving said cylindrical container in sequential clockwise and counter clockwise directions a predetermined number of revolutions to clean tissue held therein.

20. A process for cleaning bone comprising the steps of:
a) placing the bone in a perforated basket container;
b) filing the basket container with a cleaning fluid;
c) ramping up the acceleration rotational speed of the basket container prior to obtaining a desired rpm;
d) rotating the basket container in a first direction at a specified rotational speed for a specified number of rotations;
e) ramping down the deceleration rotational speed and stopping rotation;
f) ramping up the acceleration rotational speed of the basket container in an opposite direction prior to obtaining a desired rpm;
g) rotating the basket container in an opposite direction from said first direction at a specified speed for a specified number of rotations; and
e) ramping down the deceleration rotational speed after obtaining a desired rpm and stopping rotation.

21. A process as claimed in claim 20 wherein said desired rpm is about 1000 rpm.

22. A process as claimed in claim 20 wherein said number of rotations ranges between about 400 to about 500 rotations.

23. A process as claimed in claim 19 wherein said cleaning fluid is detergent.
